(19) United States
(12) Patent Application Publication
(10) Pub. No.: US 2020/0206146 A1
(43) Pub. Date: Jul. 2, 2020

(54) Medical cold compress patch and preparation method

(71) Applicant: SHANDONG ZHUSHI PHARMACEUTICAL GROUP CO., LTD., Heze City (CN)

(72) Inventors: KUNFU ZHU, HEZE CITY (CN); LEI ZHU, HEZE CITY (CN)

(21) Appl. No.: 16/550,440

(22) Filed: Aug. 26, 2019

(30) Foreign Application Priority Data
Dec. 28, 2018 (CN) 2018116232350

(51) Int. Cl. A61K 9/70; A61K 38/18; A61K 31/164; A61K 36/233; A61K 36/288; A61K 36/756; A61K 36/886; A61K 36/355; A61K 47/36; A61K 47/38; A61K 47/10

(52) U.S. Cl. CPC A61K 9/703; A61K 38/1808; A61K 31/164; A61K 36/233; A61K 36/288; A61K 47/10; A61K 36/886; A61K 36/355; A61K 47/36; A61K 47/38; A61K 36/756

(57) ABSTRACT

A medical cold compress patch includes a backing layer, a gel layer and a covering layer. The gel layer includes various raw materials, and each raw material is measured as following mass percentage, 2 to 15 mass percent of macromolecule substance, 2 to 10 mass percent of epidermal growth factor, 2 to 8 mass percent of ceramide, 3 to 8 mass percent of humectant, 3 to 10 mass percent of bupleurum sinensis extract, 2 to 8 mass percent of dandelion extract, 3 to 12 mass percent of cortex phellodendri extract, 5 to 10 mass percent of aloe vera extract, 2 to 10 mass percent of honeysuckle flowers extract and the balance being purified water. A method for preparing medical cold compress patch is also disclosed.

### TECHNICAL FIELD

The present invention relates to a technical field of medical supplies, particular to a medical cold compress patch and preparation method thereof.

### BACKGROUND ART

In traditional cold compress physiotherapy methods ice cubes or ice cube composites (commonly referred to as ice packs) are generally applied to the affected area or specific parts so that the purposes of cold compress and pain relief are achieved, but they are of short cold application time, large consumption, and uneven cold effect. When they are applied to infants or certain special parts, patients are prone to be uncomfortable and even get localized frostbite. At the same time, those products are inconvenient to carry and could be used at limited time or place, and the person must maintain a certain posture and could not move when using.

The cold compress patch obtained by improving the traditional cold compress method (like cold water, ice water, alcohol, etc.) and centered on a new drug release system TDDS which is prepared on the basis of polymer gel, comprises a backing layer, a gel layer, and a covering layer. The water and natural cooling component contained in the polymer gel vaporize to absorb the heat so that localized cooling is achieved, and the drug therein combined with the hydrogel allows the ingredients to penetrate the fatty layer quickly through the hydration, permeate into the subcutaneous tissue, directly reach the lesion site, and act on the affected area thereby achieving the effects of cold compress, percutaneous absorption and sustained release of the drug.

Ordinary cold compress patch is mainly suitable for early local soft tissue injury, high-heat patients and heatstroke, toothache and other people who need cold compress, but fails to quickly repair skin tissue and promote wound healing, in addition, due to the simple size and shape, ordinary cold compress could not meet the needs of different users.

### SUMMARY OF INVENTION

The present invention aims to solve the technical issues mentioned above and provide a new medical cold compress patch which is safe, efficient and easy to use, and the preparation method thereof.

The technical solutions adopted in present invention are as follows: The present invention provides a medical cold compress patch, comprising a backing layer, a gel layer, and a covering layer, the gel layer is comprised of various raw materials and each raw material is measured as following mass percentage, 2 to 15 mass percent of macromolecule substance, 2 to 10 mass percent of epidermal growth factor, 2 to 8 mass percent of ceramide, 3 to 8 mass percent of humectant, 3 to 10 mass percent of bupleurum sinensis extract, 2 to 8 mass percent of dandelion extract, 3 to 12 mass percent of cortex phellodendri extract, 5 to 10 mass percent of aloe vera extract, 2 to 10 mass percent of honeysuckle flowers extract and the balance being purified water.

Preferentially, the gel layer is comprised of various raw materials and each raw material is measured as following mass percentage, 5 to 10 mass percent of macromolecule substance, 5 to 8 mass percent of epidermal growth factor, 4 to 8 mass percent of ceramide, 3 to 8 mass percent of humectant, 5 to 8 mass percent of bupleurum sinensis extract, 4 to 6 mass percent of dandelion extract, 5 to 10 mass percent of cortex phellodendri extract, 7 to 9 mass percent of aloe vera extract, 4 to 7 mass percent of honeysuckle flowers extract and the balance being purified water.

Preferentially, the gel layer is comprised of various raw materials and each raw material is measured as following mass percentage, 10 mass percent of macromolecule substance, 6 mass percent of epidermal growth factor, 5 mass percent of ceramide, 4 mass percent of humectant, 6 mass percent of bupleurum sinensis extract, 5 mass percent of dandelion extract, 7 mass percent of cortex phellodendri extract, 8 mass percent of aloe vera extract, 6 mass percent of honeysuckle flowers extract and the balance being purified water.

Preferentially, the macromolecule substance comprises one or more mixtures selected from the group of polyvinyl alcohol, sodium polyacrylate, carbomer, hydroxyl propyl methyl cellulose sodium, and chitosan.

Preferentially, the humectant comprises one or more mixtures selected from the group of glycerin, sodium hyaluronate, aloe vera barbadensis leaf juice, and sorbitol.

Preferentially, the ceramide is a light brown liquid with a PH value of 5.0 to 6.0 and a total bacterial count of less than 100 cfu/g.

Preferentially, the total bacterial count in the purified water is less than 100 cfu/ml.

Preferentially, the backing layer is made of nonwoven or composite nonwoven fabrics and the covering layer is one of polyethylene film layer, polypropylene film layer and polycarbonate film layer.

Preferentially, the medical cold compress patch is configured to be a shape of split facial mask, a shape similar to female sanitary pad and a shape of rain drop.

Further, the preparation method of medical cold compress patch in accordance to any of claims 1-3, characterized in comprising the following steps of:

Step A: accurately weighing and preparing each raw material for use according to the ratios of each raw material in the gel layer;
Step B: dissolving the prepared epidermal growth factor, ceramide, humectant and aloe vera extract in water and at normal temperature stirring the solution evenly at a rate of 150-300 r/min for 15-30 min to obtain the constituent 1;
Step C: dissolving the prepared macromolecule substance in water to swell the macromolecule substance at normal temperature for 20 min and stirring the solution evenly at a rate of 50-100 r/min for 10-20 min to obtain the constituent 2;
Step D: dissolving prepared the bupleurum sinensis extract, dandelion extract, cortex phellodendri extract, aloe vera extract, and honeysuckle flowers extract in water and at normal temperature stirring the solution evenly at a rate of 100-200 r/min for 20-40 min to obtain the constituent 3;
Step E: mixing above-mentioned three constituents and at normal temperature stirring the mixture evenly at a rate of 100-200 r/min for 20-40 min to obtain a clear gel; and
Step F: coating the clear gel evenly on a backing layer and pressing a covering layer on the clear gel for tailoring, solidifying and packaging prior to obtain the finished product of the medical cold compress patch.

Preferentially, in Step B the stirring rate is 260 r/min and the stirring time is 20 min;

Preferentially, in Step C the stirring rate is 80 r/min and the stirring time is 14 min;
Preferentially, in Step D the stirring rate is 180 r/min and the stirring time is 30 min;
Preferentially, in Step E the stirring rate is 220 r/min and the stirring time is 45 min;
The advantageous effects of present invention are as follows:
1. The medical cold compress patch in the present invention is so easy to use that could be used under various occasions and applied to various parts of human body without affecting human body movement at all;
2. The medical cold compress patch in the present invention is not only cold compressed for physical cooling and relieving pain, but also contains components like epidermal growth factor to offer nutrition for the skin and promote tissue healing and recovery, the discomfort after surgery is relieved and meanwhile the skin recovery time is shortened.
3. The medical cold compress patch in the present invention is biocompatible with the skin, soft and comfortable to touch. The hydrophilic polymer matrix has the advantages like moisture retention, air permeability, sweat resistance, no sensitization and no irritation
4. The medical cold compress patch in the present invention has good drug release performance, strong affinity with the skin, and could improve the hydration of the cuticle layer, which is conducive to drug transdermal absorption. In addition, the evaporation of water and components in the gel layer is slow and continuous, and the effect time after applying is over 12 hours.
5. The medical cold compress patch in the present invention is of various shapes and could meet needs of different users.
6. The preparation method of medical cold compress patch in the present invention is of simple operation, extensive source of raw materials, low cost, no poison and no-side effects, and is safe and reliable for application.

### DESCRIPTION OF THE DRAWINGS

FIG. 1 is a schematic diagram showing the structure of the present invention, 1-covering layer, 2-hydrogel and 3-backing gel.

FIG. 2 is a schematic diagram showing a shape of split facial mask;

FIG. 3 is a schematic diagram showing a shape similar to a sanitary pad;

FIG. 4 is a schematic diagram showing a shape of a rain drop;

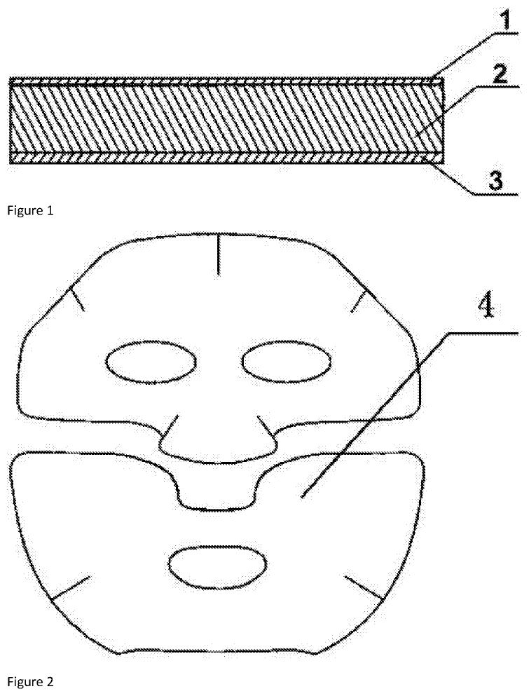

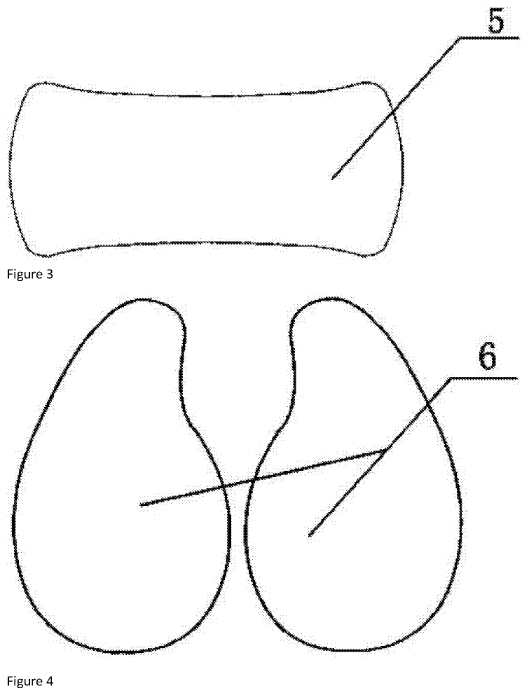

### SPECIFIC EMBODIMENTS

### Embodiment 1

A medical cold compress patch is provided, comprising a backing layer, a gel layer, and a covering layer, the gel layer is comprised of various raw materials and each raw material is measured as following mass percentage, 4 mass percent of macromolecule substance, 3 mass percent of epidermal growth factor, 2 mass percent of ceramide, 4 mass percent of humectant, 4 mass percent of bupleurum sinensis extract, 2 mass percent of dandelion extract, 4 mass percent of cortex phellodendri extract, 6 mass percent of aloe vera extract, 3 mass percent of honeysuckle flowers extract and the balance being purified water.

The macromolecule substance comprises a mixture of polyvinyl alcohol and carbomer.

The humectant is glycerin.

The ceramide is a light brown liquid with a PH value of 5.0 to 6.0 and a total bacterial count of less than 100 cfu/g.

The total bacterial count in the purified water is less than 100 cfu/ml.

The backing layer is made of nonwoven fabrics and the covering layer is polyethylene film layer.

The medical cold compress patch is in shape of rain drop.

The preparation method of medical cold compress patch comprises the following steps of:

Step A: accurately weighing and preparing each raw material for use according to the ratios of each raw material in the gel layer;
Step B: dissolving the prepared epidermal growth factor, ceramide, humectant and aloe vera extract in water and at normal temperature stirring the solution evenly at a rate of 180 r/min for 20 min to obtain the constituent 1;
Step C: dissolving the prepared macromolecule substance in water to swell the macromolecule substance at normal temperature for 20 min and stirring the solution evenly at a rate of 60 r/min for 13 min to obtain the constituent 2;
Step D: dissolving prepared the bupleurum sinensis extract, dandelion extract, cortex phellodendri extract, aloe vera extract, and honeysuckle flowers extract in water and at normal temperature stirring the solution evenly at a rate of 120 r/min for 28 min to obtain the constituent 3;
Step E: mixing above-mentioned three constituents and at normal temperature stirring the mixture evenly at a rate of 180 r/min for 40 min to obtain a clear gel; and
Step F: coating the clear gel evenly on a backing layer and pressing a covering layer on the clear gel for tailoring, solidifying and packaging prior to obtain the finished product of the medical cold compress patch

### Embodiment 2

A medical cold compress patch is provided, comprising a backing layer, a gel layer, and a covering layer, the gel layer is comprised of various raw materials and each raw material is measured as following mass percentage, 4 mass percent of macromolecule substance, 3 mass percent of epidermal growth factor, 2 mass percent of ceramide, 4 mass percent of humectant, 4 mass percent of bupleurum sinensis extract, 2 mass percent of dandelion extract, 4 mass percent of cortex phellodendri extract, 6 mass percent of aloe vera extract, 3 mass percent of honeysuckle flowers extract and the balance being purified water.

The macromolecule substance is a mixture of polyvinyl alcohol and carbomer.

The humectant is glycerin.

The ceramide is a light brown liquid with a PH value of 5.0 to 6.0 and a total bacterial count of less than 100 cfu/g.

The total bacterial count in the purified water is less than 100 cfu/ml.

The backing layer is made of nonwoven fabrics and the covering layer is polyethylene film layer.

The medical cold compress patch is in a shape of rain drop.

The preparation method of medical cold compress patch comprises the following steps of:

Step A: accurately weighing and preparing each raw material for use according to the ratios of each raw material in the gel layer;
Step B: dissolving the prepared epidermal growth factor, ceramide, humectant and aloe vera extract in water and at normal temperature stirring the solution evenly at a rate of 260 r/min for 20 min to obtain the constituent 1;
Step C: dissolving the prepared macromolecule substance in water to swell the macromolecule substance at normal temperature for 20 min and stirring the solution evenly at a rate of 80 r/min for 14 min to obtain the constituent 2;
Step D: dissolving prepared the bupleurum sinensis extract, dandelion extract, cortex phellodendri extract, aloe vera extract, and honeysuckle flowers extract in water and at normal temperature stirring the solution evenly at a rate of 180 r/min for 30 min to obtain the constituent 3;
Step E: mixing above-mentioned three constituents and at normal temperature stirring the mixture evenly at a rate of 220 r/min for 45 min to obtain a clear gel; and
Step F: coating the clear gel evenly on a backing layer and pressing a covering layer on the clear gel for tailoring, solidifying and packaging prior to obtain the finished product of the medical cold compress patch

### Embodiment 3

A medical cold compress patch is provided, comprising a backing layer, a gel layer, and a covering layer, the gel layer is comprised of various raw materials and each raw material is measured as following mass percentage, 10 mass percent of macromolecule substance, 8 mass percent of epidermal growth factor, 8 mass percent of ceramide, 8 mass percent of humectant, 8 mass percent of bupleurum sinensis extract, 6 mass percent of dandelion extract, 10 mass percent of cortex phellodendri extract, 9 mass percent of aloe vera extract, 7 mass percent of honeysuckle flowers extract and the balance being purified water.

The macromolecule substance is a mixture of sodium polyacrylate and chitosan.

The humectant comprises a mixture of sodium hyaluronate and aloe vera barbadensis leaf juice.

The ceramide is a light brown liquid with a PH value of 5.0 to 6.0 and a total bacterial count of less than 100 cfu/g.

The total bacterial count in the purified water is less than 100 cfu/ml.

The backing layer is made of nonwoven fabrics and, the covering layer is polycarbonate film layer.

The medical cold compress patch is in a shape similar to female sanitary pad.

The preparation method of medical cold compress patch comprises the following steps of:

Step A: accurately weighing and preparing each raw material for use according to the ratios of each raw material in the gel layer;
Step B: dissolving the prepared epidermal growth factor, ceramide, humectant and aloe vera extract in water and at normal temperature stirring the solution evenly at a rate of 260 r/min for 30 min to obtain the constituent 1;
Step C: dissolving the prepared macromolecule substance in water to swell the macromolecule substance at normal temperature for 20 min and stirring the solution evenly at a rate of 80 r/min for 20 min to obtain the constituent 2;
Step D: dissolving prepared the bupleurum sinensis extract, dandelion extract, cortex phellodendri extract, aloe vera extract, and honeysuckle flowers extract in water and at normal temperature stirring the solution evenly at a rate of 160-200 r/min for 36 min to obtain the constituent 3;
Step E: mixing above-mentioned three constituents and at normal temperature stirring the mixture evenly at a rate of 290 r/min for 40 min to obtain a clear gel; and
Step F: coating the clear gel evenly on a backing layer and pressing a covering layer on the clear gel for tailoring, solidifying and packaging prior to obtain the finished product of the medical cold compress patch

### Embodiment 4

A medical cold compress patch is provided, comprising a backing layer, a gel layer, and a covering layer, the gel layer is comprised of various raw materials and each raw material is measured as following mass percentage, 10 mass percent of macromolecule substance, 8 mass percent of epidermal growth factor, 8 mass percent of ceramide, 8 mass percent of humectant, 8 mass percent of bupleurum sinensis extract, 6 mass percent of dandelion extract, 10 mass percent of cortex phellodendri extract, 9 mass percent of aloe vera extract, 7 mass percent of honeysuckle flowers extract and the balance being purified water.

The macromolecule substance comprises a mixture of sodium polyacrylate and chitosan.

The humectant comprises a mixture of sodium hyaluronate and aloe vera barbadensis leaf juice.

The ceramide is a light brown liquid with a PH value of 5.0 to 6.0 and a total bacterial count of less than 100 cfu/g.

The total bacterial count in the purified water is less than 100 cfu/ml.

The backing layer is made of nonwoven fabrics and, the covering layer is polycarbonate film layer.

The medical cold compress patch is similar to a shape similar to female sanitary pad.

The preparation method of medical cold compress patch comprises the following steps of:

Step A: accurately weighing and preparing each raw material for use according to the ratios of each raw material in the gel layer;
Step B: dissolving the prepared epidermal growth factor, ceramide, humectant and aloe vera extract in water and at normal temperature stirring the solution evenly at a rate of 260 r/min for 20 min to obtain the constituent 1;
Step C: dissolving the prepared macromolecule substance in water to swell the macromolecule substance at normal temperature for 20 min and stirring the solution evenly at a rate of 80 r/min for 14 min to obtain the constituent 2;
Step D: dissolving prepared the bupleurum sinensis extract, dandelion extract, cortex phellodendri extract, aloe vera extract, and honeysuckle flowers extract in water and at normal temperature stirring the solution evenly at a rate of 180 r/min for 30 min to obtain the constituent 3;
Step E: mixing above-mentioned three constituents and at normal temperature stirring the mixture evenly at a rate of 220 r/min for 45 min to obtain a clear gel; and
Step F: coating the clear gel evenly on a backing layer, then pressing a covering layer on the clear gel and obtaining the finished product of a medical cold compress patch after tailoring, solidifying and packaging the layers.

### Embodiment 5

A medical cold compress patch is provided, comprising a backing layer, a gel layer, and a covering layer, the gel layer is comprised of various raw materials and each raw material is measured as following mass percentage, 10 mass percent of macromolecule substance, 6 mass percent of epidermal growth factor, 5 mass percent of ceramide, 4 mass percent of humectant, 6 mass percent of bupleurum sinensis extract, 5 mass percent of dandelion extract, 7 mass percent of cortex phellodendri extract, 8 mass percent of aloe vera extract, 6 mass percent of honeysuckle flowers extract and the balance being purified water.

The macromolecule substance comprises a mixture of carbomer, hydroxyl propyl methyl cellulose sodium and chitosan.

The humectant comprises a mixture of glycerin, sodium hyaluronate and aloe vera barbadensis leaf juice.

The ceramide is a light brown liquid with a PH value of 5.0 to 6.0 and a total bacterial count of less than 100 cfu/g.

The total bacterial count in the purified water is less than 100 cfu/ml.

The backing layer is made of composite nonwoven fabrics and the covering layer is polycarbonate film layer.

The medical cold compress patch is in a shape of split facial mask.

The preparation method of medical cold compress patch comprises the following steps of:

Step A: accurately weighing and preparing each raw material for use according to the ratios of each raw material in the gel layer;
Step B: dissolving the prepared epidermal growth factor, ceramide, humectant and aloe vera extract in water and at normal temperature stirring the solution evenly at a rate of 260 r/min for 20 min to obtain the constituent 1;
Step C: dissolving the prepared macromolecule substance in water to swell the macromolecule substance at normal temperature for 20 min and stirring the solution evenly at a rate of 80 r/min for 14 min to obtain the constituent 2;
Step D: dissolving prepared the bupleurum sinensis extract, dandelion extract, cortex phellodendri extract, aloe vera extract, and honeysuckle flowers extract in water and at normal temperature stirring the solution evenly at a rate of 180 r/min for 30 min to obtain the constituent 3;
Step E: mixing above-mentioned three constituents and at normal temperature stirring the mixture evenly at a rate of 220 r/min for 45 min to obtain a clear gel; and
Step F: coating the clear gel evenly on a backing layer and pressing a covering layer on the clear gel for tailoring, solidifying and packaging prior to obtain the finished product of the medical cold compress patch.

### Embodiment 6

A medical cold compress patch is provided, comprising a backing layer, a gel layer, and a covering layer, the gel layer is comprised of various raw materials and each raw material is measured as following mass percentage, 10 mass percent of macromolecule substance, 6 mass percent of epidermal growth factor, 5 mass percent of ceramide, 4 mass percent of humectant, 6 mass percent of bupleurum sinensis extract, 5 mass percent of dandelion extract, 7 mass percent of cortex phellodendri extract, 8 mass percent of aloe vera extract, 6 mass percent of honeysuckle flowers extract and the balance being purified water.

The macromolecule substance comprises a mixture of sodium carbomer, hydroxyl propyl methyl cellulose sodium and chitosan.

The humectant is a mixture of glycerin, sodium hyaluronate and aloe vera barbadensis leaf juice.

The ceramide is a light brown liquid with a PH value of 5.0 to 6.0 and a total bacterial count of less than 100 cfu/g.

The total bacterial count in the purified water is less than 100 cfu/ml.

The backing layer is made of composite nonwoven fabrics and, the covering layer comprises polycarbonate film layer.

The medical cold compress patch is in a shape of split facial mask.

The preparation method of medical cold compress patch comprises the following steps of:

Step A: accurately weighing and preparing each raw material for use according to the ratios of each raw material in the gel layer;
Step B: dissolving the prepared epidermal growth factor, ceramide, humectant and aloe vera extract in water and at normal temperature stirring the solution evenly at a rate of 180 r/min for 20 min to obtain the constituent 1;
Step C: dissolving the prepared macromolecule substance in water to swell the macromolecule substance at normal temperature for 20 min and stirring the solution evenly at a rate of 60 r/min for 13 min to obtain the constituent 2;
Step D: dissolving prepared the bupleurum sinensis extract, dandelion extract, cortex phellodendri extract, aloe vera extract, and honeysuckle flowers extract in water and at normal temperature stirring the solution evenly at a rate of 120 r/min for 28 min to obtain the constituent 3;
Step E: mixing above-mentioned three constituents and at normal temperature stirring the mixture evenly at a rate of 180 r/min for 40 min to obtain a clear gel; and
Step F: coating the clear gel evenly on a backing layer and pressing a covering layer on the clear gel for tailoring, solidifying and packaging prior to obtain the finished product of the medical cold compress patch

The specific embodiments of the present invention are only a part of the embodiments of the present invention, and are not intended to limit the invention. All other embodiments obtained by those skilled in the art based on the embodiments of the present invention without creative efforts are within the scope of the present invention.

1. A medical cold compress patch comprising a backing layer, a gel layer, and a covering layer, characterized in that the gel layer is consisted of various raw materials and each raw material is measured by mass percentage, 2 to 15 mass percent of macromolecule substance, 2 to 10 mass percent of epidermal growth factor, 2 to 8 mass percent of ceramide, 3 to 8 mass percent of humectant, 3 to 10 mass percent of bupleurum sinensis extract, 2 to 8 mass percent of dandelion extract, 3 to 12 mass percent of cortex phellodendri extract, 5 to 10 mass percent of aloe vera extract, 2 to 10 mass percent of honeysuckle flowers extract and the balance being purified water.

2. The medical cold compress patch in accordance with claim 1, comprising a backing layer, a gel layer, and a covering layer, characterized in that the gel layer is comprised of various raw materials and each raw material is measured as the following mass percentage, 5 to 10 mass percent of macromolecule substance, 5 to 8 mass percent of epidermal growth factor, 4 to 8 mass percent of ceramide, 3 to 8 mass percent of humectant, 5 to 8 mass percent of bupleurum sinensis extract, 4 to 6 mass percent of dandelion extract, 5 to 10 mass percent of cortex phellodendri extract, 7 to 9 mass percent of aloe vera extract, 4 to 7 mass percent of honeysuckle flowers extract and the balance being purified water.

3. The medical cold compress patch in accordance with claim 1, comprising a backing layer, a gel layer, and a covering layer, characterized in that the gel layer is comprised of various raw materials and each raw material is measured as following mass percentage, 10 mass percent of macromolecule substance, 6 mass percent of epidermal growth factor, 5 mass percent of ceramide, 4 mass percent of humectant, 6 mass percent of bupleurum sinensis extract, 5 mass percent of dandelion extract, 7 mass percent of cortex phellodendri extract, 8 mass percent of aloe vera extract, 6 mass percent of honeysuckle flowers extract and the balance being purified water.

4. The medical cold compress patch in accordance with claim 1, characterized in that the macromolecule substance comprises one or more mixtures selected from the group of polyvinyl alcohol, sodium polyacrylate, carbomer, hydroxyl propyl methyl cellulose sodium, and chitosan.

5. The medical cold compress patch in accordance with claim 1, characterized in that the humectant comprises one or more mixtures selected from the group of glycerin, sodium hyaluronate, aloe vera barbadensis leaf juice, and sorbitol.

6. The medical cold compress patch in accordance with claim 1, characterized in that the ceramide is a light brown liquid with a PH value between 5.0 to 6.0 and a total bacterial count of less than 100 cfu/g.

7. The medical cold compress patch in accordance with claim 1, characterized in that the total bacterial count in the purified water is less than 100 cfu/ml.

8. The medical cold compress patch in accordance with claim 1, characterized in that the backing layer is made of nonwoven or composite nonwoven fabrics, the covering layer is one of polyethylene film layer, polypropylene film layer and polycarbonate film layer.

9. The medical cold compress patch in accordance with claim 1, characterized in that the medical cold compress patch is configured to be a shape of split-type facial mask, a shape similar to female sanitary pad and a shape of rain drop.

10. A preparation method of the medical cold compress patch, comprising the following steps of:
Step A: accurately weighing and preparing each raw material for use according to the ratios of each raw material in the gel layer;
Step B: dissolving the prepared epidermal growth factor, ceramide, humectant and aloe vera extract in water and mixing the solution evenly at a rate of 150-300 r/min for 15-30 min at normal temperature to obtain the constituent 1;
Step C: dissolving the prepared macromolecule substance in water to swell the macromolecule substance at normal temperature for 20 min and stirring the solution evenly at a rate of 50-100 r/min for 10-20 min to obtain the constituent 2,
Step D: dissolving prepared the bupleurum sinensis extract, dandelion extract, cortex phellodendri extract, aloe vera extract, and honeysuckle flowers extract in water and at normal temperature stirring the solution evenly at a rate of 100-200 r/min for 20-40 min to obtain the constituent 3;
Step E: mixing above-mentioned three constituents and at normal temperature stirring the mixture evenly at a rate of 100-200 r/min for 20-40 min to obtain a clear gel; and
Step F: coating the clear gel evenly on a backing layer and pressing a covering layer against the clear gel and cutting, solidifying and packing it to obtain the finished product of the medical cold compress patch
